(19) United States
(12) Patent Application Publication
(10) Pub. No.: US 2026/0000807 A1
(43) Pub. Date: Jan. 1, 2026

(54) MEDICAL COMPOSITION, HEMOSTATIC AGENT, AND DISPENSER

(71) Applicant: FUJIFILM Corporation, Tokyo (JP)

(72) Inventor: Mamoru KURAMOTO, Kanagawa (JP)

(73) Assignee: FUJIFILM Corporation, Tokyo (JP)

(21) Appl. No.: 19/248,395

(22) Filed: Jun. 24, 2025

(30) Foreign Application Priority Data
Jun. 28, 2024 (JP) 2024-105754

(51) Int. Cl. A61L 24/04; A61L 24/00; A61M 11/02; A61M 25/00; A61M 39/24

(52) U.S. Cl. CPC A61L 24/043; A61L 24/0026; A61M 11/02; A61M 25/0026; A61M 39/24; A61L 2400/04; A61M 2039/2446; A61M 2202/064

(57) ABSTRACT

Provided are a medical composition and a hemostatic agent which are excellent in both hemostatic properties and retention properties over time, and a dispenser with which the medical composition or the hemostatic agent can be satisfactorily sprayed.

The medical composition is applied to a surface of an inner lumen of a digestive tract of a living body; the dispenser sprays the medical composition and the hemostatic agent, which are powder bodies containing polyacrylic acid and polyvinylpyrrolidone, and the medical composition onto the surface of the inner lumen of the digestive tract; the dispenser includes a main body and a catheter, in which the main body has an accommodation portion that accommodates air from an outside of the living body and that is configured to eject the air by pressurizing the accommodated air, a container that accommodates the medical composition, and at least two check valves; the catheter has a connecting portion that is connected to the container and through which the medical composition flows; and the dispenser is configured to spray the medical composition onto the surface of the inner lumen of the digestive tract from an end part of the catheter by ejecting the air from the accommodation portion.

### CROSS-REFERENCE TO RELATED APPLICATIONS

The present application claims priority under 35 U.S.C. § 119 to Japanese Patent Application No. 2024-105754 filed on Jun. 28, 2024. The above application is hereby expressly incorporated by reference, in its entirety, into the present application.

### BACKGROUND

### 1. Technical Field of the Invention

The present disclosure relates to a medical composition, a hemostatic agent, and a dispenser.

### 2. Description of the Related Art

In the medical field, endoscopic diagnosis or endoscopic treatment using an endoscope is widely performed. In endoscopic diagnosis or endoscopic treatment, for example, a treatment such as resection of a lesion by a treatment tool is performed on a digestive organ. For example, endoscopic submucosal dissection (ESD), endoscopic mucosal resection (EMR), or the like, which is a technique for resecting cancer tissue in a mucosal surface layer using an endoscope, is generally performed for early gastric cancer, colon cancer, and the like.

In recent years, since rebleeding may occur or postoperative perforation may occur in a resection part of a mucosal layer such as an ulcer base, it is considered desirable to protect the ulcer base, for example, by suturing with a clip. In addition, in another method, attempts have been made to protect the mucosal layer including the ulcer base by spraying a medical adhesive.

A medical two-component reactive adhesive including a mixed powder containing a powder of aldehyde glucan and a powder of partially carboxylated poly-L-lysine is known as a medical adhesive that is used for a mucosal layer or the like after resection of a lesion by endoscopic treatment, surgical operation, or the like and that is closely attached to an adherend such as an organ until a wound is healed (JP2019-092645A). JP2019-092645A describes that the medical two-component reactive adhesive can be applied to a wound or the like by spraying and can be used as a hemostatic agent.

### SUMMARY

In a case where bleeding of a digestive tract mucous membrane due to treatment or the like in endoscopic diagnosis or endoscopic treatment is stopped by a hemostatic agent spraying method, it is desired that a hemostatic agent of a type in which powders are sprayed is excellent in both hemostatic properties of performing hemostasis satisfactorily at a bleeding site and retention properties over time in which the hemostatic agent is moderately retained at the bleeding site without being dissolved or decomposed by a body fluid or the like.

The present disclosure has been made in view of the above.

An object to be achieved by an embodiment of the present disclosure is to provide a medical composition and a hemostatic agent, which are excellent in both hemostatic properties and retention properties over time, and a dispenser with which the medical composition or the hemostatic agent can be satisfactorily sprayed.

The specific means for achieving the objects includes the following aspects.
<1> A medical composition to be applied to a surface of an inner lumen of a digestive tract of a living body, in which the medical composition is a powder body containing polyacrylic acid and polyvinylpyrrolidone.
<2> The medical composition according to <1>, in which the powder body includes a powder of the polyacrylic acid and a powder of the polyvinylpyrrolidone.
<3> The medical composition according to <1> or <2>, in which particles contained in a powder of the polyacrylic acid are attached to surfaces of particles contained in a powder of the polyvinylpyrrolidone to form the powder body.
<4> The medical composition according to any one of <1> to <3>, in which the powder body is a granule containing a powder of the polyacrylic acid and a powder of the polyvinylpyrrolidone.
<5> The medical composition according to <4>, in which the granule has a volume distribution average particle diameter D50 of 50 μm to 500 μm.
<6> The medical composition according to any one of <1> to <5>, in which the polyacrylic acid and the polyvinylpyrrolidone are contained at a ratio of 1:10 to 10:1 in terms of mass ratio.
<7> The medical composition according to any one of <1> to <6>, in which a total content of the polyacrylic acid and the polyvinylpyrrolidone is 10% by mass to 100% by mass with respect to a total mass of the medical composition.
<8> The medical composition according to any one of <1> to <7>, in which the medical composition is a hemostatic agent.
<9> A hemostatic agent comprising the medical composition according to any one of <1> to <7>.
<10> A dispenser that sprays the medical composition according to any one of <1> to <8> or the hemostatic agent according to <9> onto a surface of an inner lumen of a digestive tract, the dispenser comprising: a main body; and a catheter, in which the main body has an accommodation portion that accommodates air from an outside of a living body and that is configured to eject the air by pressurizing the accommodated air, a container that accommodates the medical composition, and at least two check valves, the catheter has a connecting portion that is connected to the container and through which the medical composition flows, and the dispenser is configured to spray the medical composition onto the surface of the inner lumen of the digestive tract from an end part of the catheter by ejecting the air from the accommodation portion.
<11> The dispenser according to <10>, in which at least one of the check valves has a function of being biased in a closing direction at normal pressure and being opened in a case of pressurization.
<12> The dispenser according to <10> or <11>, in which at least one of the check valves is a duckbill check valve.
<13> The dispenser according to any one of <10> to <12>, in which the main body includes a connecting portion that connects the accommodation portion and the container, and the connecting portion is provided with at least one duckbill check valve.
<14> The dispenser according to any one of <10> to <13>, in which the accommodation portion is a spherical structure including an elastic resin configured to pressurize the air by squeezing.
<15> The dispenser according to any one of <10> to <14>, in which the catheter has an inner diameter of 0.5 mm to 2.5 mm and is disposed at a distal end of the main body.
<16> The dispenser according to any one of <10> to <15>, in which the catheter has at least two flow passages, a flow passage through which the medical composition flows and a flow passage through which water flows.

According to an embodiment of the present disclosure, there are provided a medical composition and a hemostatic agent which are excellent in both hemostatic properties and retention properties over time, and a dispenser with which the medical composition or the hemostatic agent can be satisfactorily sprayed.

### BRIEF DESCRIPTION OF THE DRAWINGS

FIG. 1 is a photograph of a powder body of a medical composition, the photograph being captured by an electron microscope (specifically, a scanning electron microscope (SEM)).

FIG. 2 is an explanatory view showing an example of a dispenser.

FIG. 3 is an explanatory view showing another example of the dispenser.

FIG. 4 is a graph showing a volume-based particle size distribution of Carbopol 971P, in which a bar graph shows a frequency distribution (left scale) and a line graph shows an integral distribution (right scale).

FIG. 5 is a graph showing a volume-based particle size distribution of PVP K90, in which a bar graph shows a frequency distribution (left scale) and a line graph shows an integral distribution (right scale).

FIG. 6 is a graph showing a volume-based particle size distribution of the medical composition obtained in Example 2, in which a bar graph shows a frequency distribution (left scale) and a line graph shows an integral distribution (right scale).

### DETAILED DESCRIPTION

Hereinafter, embodiments of the present disclosure will be described. The present disclosure is not limited in any way to the following embodiments, and may be implemented with appropriate modifications within the scope of the object of the present disclosure.

In the present disclosure, the numerical ranges shown using “to” indicate ranges including the numerical values described before and after “to” as a lower limit value and an upper limit value. Regarding the numerical ranges that are described stepwise in the present disclosure, an upper limit value or a lower limit value described in a certain numerical range may be replaced with an upper limit value or a lower limit value of another stepwise numerical range. In addition, in the numerical ranges described in the present disclosure, an upper limit value or a lower limit value described in a certain numerical range may be replaced with a value described in Examples.

In the present disclosure, the amount of each component in a composition means, in a case where the composition contains a plurality of substances corresponding to such a component, the total amount of the plurality of substances in the composition unless otherwise specified.

In the present disclosure, a combination of two or more preferred aspects or embodiments is a more preferred aspect or embodiment.

In the present disclosure, “% by mass” and “% by weight” have the same meaning, and “part(s) by mass” and “part(s) by weight” have the same meaning. In the present disclosure, “%” indicating an amount of a component contained is mass-based unless otherwise specified.

In the present disclosure, the “powder body” includes “powder” and “granule”.

In the present disclosure, the “granule” is formed of particles in a powder and has a size larger than that of a primary particle of the powder (including a secondary particle and a granulated product composed of a plurality of primary particles).

In the present disclosure, normal pressure means standard atmospheric pressure at 25° C.

In the present disclosure, the weight-average molecular weight (Mw) is a value measured by a GPC method.

Specifically, the above-mentioned molecular weight is a molecular weight detected using a gel permeation chromatography (GPC) analyzer using TSKgel GMHxL, TSKgel G4000HxL, and TSKgel G2000HxL columns (each is a trade name, manufactured by TOSOH CORPORATION), tetrahydrofuran (THF) as a solvent, and a differential refractometer, and expressed in terms of polystyrene as a standard substance.

In the present disclosure, the term “particle diameter” means an average particle diameter unless otherwise specified.

In the present disclosure, the average particle diameter is a volume distribution average particle diameter D50, and is a value measured by the following method.

The powder body particles to be measured were quantified for a particle size distribution under conditions of a measurement range of 0.10 μm to 3,500.00 μm and a compressed air pressure of 0.1 MPa in an eject type dry measurement mode using a laser diffraction/scattering type particle size distribution analyzer (LMS-3000 (manufactured by Malvern Panalytical Ltd.)).

Hereinafter, the medical composition, the hemostatic agent, and the dispenser according to the present disclosure will be described in detail.

### (Medical Composition)

A medical composition according to an embodiment of the present disclosure (hereinafter, also referred to as a medical composition) is a medical composition to be applied to a surface of an inner lumen of a digestive tract of a living body, in which the medical composition is a powder body containing polyacrylic acid and polyvinylpyrrolidone.

In a hemostatic agent of a type in which a powder is sprayed on a bleeding site of a digestive tract mucous membrane, it is desired that both hemostatic properties in which bleeding is satisfactorily stopped and retention properties over time in which the hemostatic agent is satisfactorily retained at the bleeding site without being decomposed by moisture or the like are excellent.

The present inventor has found that the medical composition, which is a powder body containing polyacrylic acid and polyvinylpyrrolidone, is applicable to the surface of the inner lumen of the digestive tract of the living body, and is a medical composition excellent in both hemostatic properties and retention properties over time.

Although the detailed mechanism is not clear, the present inventor presumes that the polyacrylic acid and the polyvinylpyrrolidone each form an aqueous solution with moisture contained in or supplied to blood, mucous membranes, or the like, and then form a hydrogen bond with each other, thereby forming a good gel as a hemostatic agent for in vivo use, and thus the hemostatic agent is excellent in both the hemostatic properties and the retention properties over time.

In addition, the powder body containing polyacrylic acid and polyvinylpyrrolidone, or the gel generated from the powder body, is a chemically stable resin, and is also excellent as a hemostatic agent from the viewpoint of extremely high safety.

The medical composition is preferably applied to the surface of the inner lumen of the digestive tract of the living body by using an endoscope. The medical composition can also be applied to a target other than the surface of the inner lumen of the digestive tract of the living body. For example, the medical composition can be applied to any site in the living body, such as a wound or the like in laparoscopic surgery, laparotomy, or the like using a rigid endoscope.

The medical composition may be any powder body containing polyacrylic acid and polyvinylpyrrolidone, and the method of synthesizing the powder body is not limited. The polyacrylic acid may include polymethacrylic acid, and may be called a carboxyvinyl polymer. In addition, the medical composition may contain a structural unit derived from a monomer different from acrylic acid, as long as the effect of the medical composition is not impaired. In addition, a part of the polyacrylic acid may form a salt of sodium, calcium, or the like. Furthermore, the polyacrylic acid may be chemically crosslinked in the molecule. Therefore, a carboxyvinyl polymer can be used as the polyacrylic acid. The polyacrylic acid and the polyvinylpyrrolidone may be each one kind or may include a plurality of kinds, and the combination thereof is not limited.

The polyacrylic acid is a polymer obtained by polymerizing acrylic acid.

Polyvinylpyrrolidone is typically a polymer obtained by polymerizing N-vinyl-2-pyrrolidone. The medical composition may contain a structural unit derived from a monomer different from N-vinyl-2-pyrrolidone, as long as the effect of the medical composition is not impaired. Specifically, a copolymer of polyvinylpyrrolidone and vinyl acetate may be used. Furthermore, the polyvinylpyrrolidone may be chemically crosslinked in the molecule.

In the medical composition, it is preferable that the polyacrylic acid and the polyvinylpyrrolidone are contained at a ratio of 1:10 to 10:1 in terms of mass ratio. More preferably, the polyacrylic acid and the polyvinylpyrrolidone are contained at a ratio of 1:3 to 2:1 in terms of mass ratio. In a case where the medical composition is applied to the surface of the inner lumen of the digestive tract of the living body with polyacrylic acid and polyvinylpyrrolidone in the above-described ranges, the medical composition is excellent in hemostatic properties in a case of being applied to a bleeding site, and is a gel that moderately remains on an application site due to moisture in the surface of the inner lumen of the digestive tract of the living body, and is excellent in retention properties over time. Thus, the medical composition is excellent in both hemostatic properties and retention properties over time, which is preferable.

In the medical composition, the total content of the polyacrylic acid and the polyvinylpyrrolidone is preferably 10% by mass to 100% by mass with respect to the total mass of the medical composition. The medical composition may contain polyacrylic acid and polyvinylpyrrolidone, or may contain or not contain raw materials other than these. In the medical composition, the total content of the polyacrylic acid and the polyvinylpyrrolidone is more preferably 50% by mass to 100% by mass.

In a case where the medical composition is applied to the surface of the inner lumen of the digestive tract of the living body with polyacrylic acid and polyvinylpyrrolidone in the above-described ranges, the medical composition is excellent in hemostatic properties in a case of being applied to a bleeding site, and is a gel that moderately remains on an application site due to moisture in the surface of the inner lumen of the digestive tract of the living body, and is excellent in retention properties over time. Thus, the medical composition is excellent in both hemostatic properties and retention properties over time, which is preferable.

In the medical composition, any component other than the polyacrylic acid and the polyvinylpyrrolidone can be used as long as the function of the medical composition is not significantly impaired. Examples thereof include a drug for a bleeding site, a component such as hyaluronic acid that improves absorption with a living body, and a colorant that improves the identifiability of a sprayed site. In addition, the powder component can also contain moisture in an amount of less than 20%. The medical composition may contain moisture in a process of forming secondary particles described later, and in this case, the medical composition may contain moisture in an amount of less than 20% with respect to the total mass of the medical composition.

In a case where the hemostatic properties are excellent, although it depends on the bleeding situation, for example, in a case where the medical composition is sprayed onto the bleeding site, hemostasis is achieved within a certain period of time such as 10 minutes.

In addition, in a case where the medical composition has excellent retention properties over time, although it depends on the conditions of the site on which the medical composition is sprayed, for example, in a case where the medical composition is sprayed onto the bleeding site, the medical composition adheres to the sprayed site for a certain period of time such as several hours, several days, or more.

The method of producing the powder body containing polyacrylic acid and polyvinylpyrrolidone is not limited. The powder body containing polyacrylic acid and polyvinylpyrrolidone preferably has an average particle diameter in a range of 10.0 μm to 1,000 μm, and more preferably in a range of 50.0 μm to 500 μm. In a case where the average particle diameter of the powder body is 50.0 μm or more, the powder body is easy to handle, and, for example, in a case where the powder body is sprayed using an endoscope, scattering of the powder is limited. Thus, it is possible to suppress fogging of the endoscopic visual field, which is preferable. On the other hand, in a case where the average particle diameter of the powder body is smaller than 500 μm, the occurrence of clogging of a catheter or the like used in a case of being sprayed by an endoscope is suppressed, which is preferable.

As a method of adjusting the average particle diameter of the powder body containing polyacrylic acid and polyvinylpyrrolidone, a known method can be adopted, and, for example, the average particle diameter can be adjusted by granulating the raw materials.

The powder body containing polyacrylic acid and polyvinylpyrrolidone preferably contains a powder of polyacrylic acid and a powder of polyvinylpyrrolidone. The powder of polyacrylic acid and the powder of polyvinylpyrrolidone may be in the form of powder, respectively. By mixing these powders, the powder body containing polyacrylic acid and polyvinylpyrrolidone can be obtained. The mixing is not particularly limited in terms of uniformity, and may be performed by using a known mixer. The powder of polyacrylic acid and the powder of polyvinylpyrrolidone may be each one kind or may include a plurality of kinds, and the combination thereof is not limited.

As the powder of polyacrylic acid, any powder can be used as long as it is a powder. For example, the powder of a polyacrylic acid having a weight-average molecular weight (Mw) of 5,000 or more is preferably used, and the powder of a polyacrylic acid having a weight-average molecular weight of 40,000 or more or having an intramolecular crosslinking structure is more preferable. In addition, for example, an average particle diameter of 1 μm to 200 μm is preferably used, and an average particle diameter of 1 μm to 10 μm is more preferable.

As the powder of polyvinylpyrrolidone, any powder can be used as long as it is a powder. For example, the powder of polyvinylpyrrolidone having a weight-average molecular weight (Mw) of 5,000 to 3,500,000 is preferably used, and 300,000 to 2,000,000 is more preferable. In addition, for example, an average particle diameter of 10 μm to 200 μm is preferably used, and an average particle diameter of 100 μm to 200 μm is more preferable.

The average particle diameter of the primary particles in the powder of polyacrylic acid and the average particle diameter of the primary particles in the powder of polyvinylpyrrolidone can be used without limitation. The average particle diameters of the two may be the same, or one may have a larger average particle diameter than the other.

The average particle diameter of the primary particles in the powder of polyacrylic acid and the average particle diameter of the primary particles in the powder of polyvinylpyrrolidone may be used after each of the average particle diameters are adjusted. As a method of adjusting the average particle diameter, in each primary particle, for example, it is possible to reduce the particle diameter, increase the particle diameter, and adjust the particle diameter distribution.

As a method of adjusting the average particle diameter, a known method can be adopted. For example, the powder body containing polyacrylic acid and polyvinylpyrrolidone may be prepared by performing in advance a treatment of increasing a particle diameter with granulation on primary particles in the powder of polyacrylic acid, performing in advance a treatment of further reducing a particle diameter by stirring or the like on primary particles in the powder of polyvinylpyrrolidone, and mixing the particles.

As the powder of polyacrylic acid and the powder of polyvinylpyrrolidone, for example, each of commercially available powders can be used. Examples of the powder of polyacrylic acid include Carbopol 971P manufactured by Lubrizol Corporation and HIVISWAKO 104 manufactured by FUJIFILM Wako Pure Chemical Corporation, and examples of the powder of polyvinylpyrrolidone include Kollidon F90EVO manufactured by BASF SE and Plasdon K90 manufactured by Ashland Inc., and these can be used.

The average particle diameter of the powder body, which is the granulated secondary particle, may be adjusted by performing granulation using the powder of polyacrylic acid and the powder of polyvinylpyrrolidone as raw materials. As a method of adjusting the average particle diameter of the powder body of the secondary particles, a known method can be adopted, and, for example, the average particle diameter of the powder body, which is the secondary particle, can be adjusted by performing granulation by fluidized bed granulation using, for example, a powder of polyacrylic acid and a powder of polyvinylpyrrolidone as raw materials.

It is preferable that the powder body containing polyacrylic acid and polyvinylpyrrolidone is formed by the particles of the powder of polyacrylic acid being attached to the surface of the particles of the powder of polyvinylpyrrolidone. In this case, it is preferable that the average particle diameter of the primary particles of the powder of polyvinylpyrrolidone is larger than the average particle diameter of the primary particles of the powder of polyacrylic acid. For example, a powder of primary particles of polyvinylpyrrolidone having an average particle diameter of 50 μm to 150 μm and a powder of primary particles of polyacrylic acid having an average particle diameter of 0.5 μm to 10 μm can be used as raw materials. The powder body containing polyacrylic acid and polyvinylpyrrolidone may include, for example, secondary particles formed by attaching primary particles of the plurality of polyacrylic acid powders to one primary particle of a powder of polyvinylpyrrolidone.

A method of producing a powder body of secondary particles in which the primary particles of the powder of polyacrylic acid are attached to the surface of the primary particles of the powder of polyvinylpyrrolidone is not limited, but, for example, a powder body, which is formed by attaching the particles of the powder of the polyacrylic acid to the surface of the particles of the powder of the polyvinylpyrrolidone, can be prepared by performing fluidized bed granulation by adding water using the powder of polyacrylic acid and the powder of polyvinylpyrrolidone as raw materials.

Both the powder of polyacrylic acid and the powder of polyvinylpyrrolidone are water-soluble, and, for example, by using water, the two can be easily bound to each other, and the powder body formed by attaching the particles of the powder of the polyacrylic acid to the surface of the particles of the powder of the polyvinylpyrrolidone can be obtained. In addition, the average particle diameter and the like of the powder body of the secondary particles can be adjusted by adjusting the amount of each of the powder of polyacrylic acid, the powder of polyvinylpyrrolidone, and water, which are used as raw materials, adjusting the timing of adding water, adjusting the granulation treatment such as adjustment of the granulation rate, and the like.

It is possible to suppress the occurrence of the following problems by adjusting the average particle diameter of the secondary particles of the powder body formed by attaching the particles of the powder of the polyacrylic acid to the surface of the particles of the powder of the polyvinylpyrrolidone. For example, in a case of spraying the medical composition, which is the powder body containing polyacrylic acid and polyvinylpyrrolidone, on the surface of the inner lumen of the digestive tract, it is possible to suppress fogging of the endoscopic visual field due to the scattering of the powder, and in a case of transporting the medical composition to a sprayed site, it is possible to suppress clogging of the catheter.

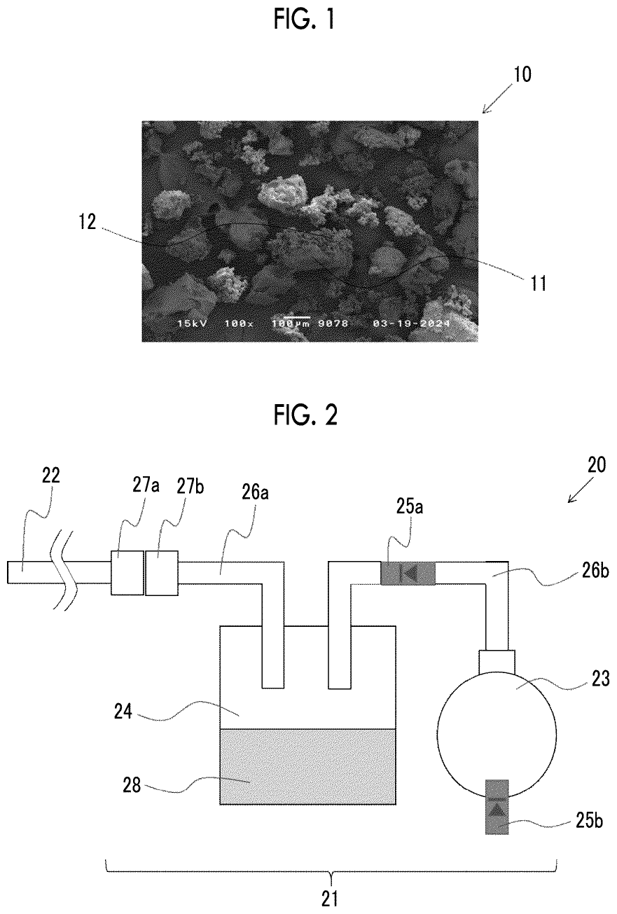

As shown in FIG. 1, in a photograph 10 captured by an electron microscope (scanning electron microscope, SEM) that images the powder body of the medical composition, the powder body formed by attaching particles 12 of the powder of the polyacrylic acid to the surface of particles 11 of the powder of the polyvinylpyrrolidone is a powder body in which the particles 12 of the powder of the polyacrylic acid are bound to the particles 11 of the powder of the polyvinylpyrrolidone. In the photograph 10, the surface of the powder body of the medical composition has fine unevenness due to binding of a large number of the particles 12 of the fine powder of polyacrylic acid to the particles 11 of the powder of polyvinylpyrrolidone, and a large amount of such powder is included. In addition, in a powder body formed by attaching particles 12 of a powder of polyacrylic acid to a surface of particles 11 of a powder of polyvinylpyrrolidone, the average particle diameter is 100 μm to 200 μm. The powder body shown in FIG. 1 is prepared by adding water to a powder of polyacrylic acid and a powder of polyvinylpyrrolidone and performing fluidized bed granulation.

The powder body containing polyacrylic acid and polyvinylpyrrolidone is preferably a granule containing a powder of polyacrylic acid and a powder of polyvinylpyrrolidone. The granule may be a granule containing a powder of polyacrylic acid and a powder of polyvinylpyrrolidone, and may be a granule granulated from these powders.

The average particle diameter of the granule is preferably 50 μm to 500 μm and more preferably 100 μm to 300 μm. It is preferable that the particle size distribution of the granule is unimodal.

In a case where the average particle diameter of the granule is within the above-described ranges, in a case of spraying the medical composition, which is the powder body containing polyacrylic acid and polyvinylpyrrolidone, on the surface of the inner lumen of the digestive tract, for example, it is possible to suppress fogging of the endoscopic visual field, and in a case of transporting the medical composition to the sprayed site, for example, it is possible to suppress the clogging of the catheter. Therefore, it is possible to suppress the occurrence of the following problems, which is preferable. In addition, in a case of forming the granule, it is easy to control the state of the powder body, such as adjusting the average particle diameter, and thus it is possible to use the powder body according to the type of the dispenser that sprays the medical composition.

A method of preparing the granule containing the powder of polyacrylic acid and the powder of polyvinylpyrrolidone is not limited, but, for example, the granule can be prepared by performing a granulation treatment on the powder of polyacrylic acid and the powder of polyvinylpyrrolidone. Both the powder of polyacrylic acid and the powder of polyvinylpyrrolidone are water-soluble, and, for example, with water, both can be easily bound to each other to form the granule. In addition, the particle size distribution, the average particle diameter, the specific surface area, and the like of the granule, which is the secondary particle, can be adjusted by adjusting the amount of each of the powder of polyacrylic acid, the powder of polyvinylpyrrolidone, and water, which are used as raw materials, adjusting the timing of adding water, adjusting the granulation treatment such as adjustment of the granulation rate, and the like.

It is preferable that the secondary particles have a large specific surface area and are bulky particles. In a case where the specific surface area is large, contact with moisture is easy, and thus rapid dissolution after spraying can be expected. In addition, in a case where the secondary particles are bulky, the storage is expected in a state where the particles are less likely to be compacted due to vibration or the like during transportation, and the spraying is easily performed.

It is preferable that the granule containing the powder of polyacrylic acid and the powder of polyvinylpyrrolidone are formed by the particles of the powder of polyacrylic acid being attached to the surface of the particles of the powder of polyvinylpyrrolidone. For example, a granule which is a secondary particle can be obtained by performing fluidized bed granulation by using the powder of polyvinylpyrrolidone having the average particle diameter of primary particles of 50 μm to 150 μm and the powder of polyacrylic acid having the average particle diameter of primary particles of 0.5 μm to 10 μm as raw materials and adding water thereto. The powder body shown in FIG. 1 is a granule formed by attaching the particles of the powder of the polyacrylic acid to the surface of the particles of the powder of the polyvinylpyrrolidone.

The method of producing the medical composition is not particularly limited, but, for example, as described above, a method of granulation with the powder of polyacrylic acid, the powder of polyvinylpyrrolidone, and water is preferable.

It is noted that the water used in this case is preferably water containing a component such as a colorant. For example, in a case where the granule using water containing a component such as a colorant is sprayed on the bleeding site, the sprayed site is colored, and the sprayed site can be made to be conspicuous.

As the colorant, any colorant that does not have a bad effect on the living body can be used. In addition, since the colorant is sprayed in the living body, a color that can be conspicuous in a case of being sprayed to red, such as blue or green, is preferable. Specifically, a coloring agent generally sprayed in a colorant method, such as a food coloring, a coloring agent in endoscopy, or a coloring agent in endoscopic treatment can be used.

Specific examples of the food coloring include tartrazine coloring agents such as Fast Green FCF (Green 3), Brilliant Blue FCF (Blue 1), and indigo carmine (Blue 2). In endoscopy or endoscopic treatment, specific examples of the coloring agent generally sprayed in the colorant method include indigo carmine, crystal violet (Pyoktanin), and methylene blue. Lugol's iodine solution, toluidine blue, and the like, which are used in an esophagus, are also used depending on the procedure of endoscopy or endoscopic treatment. In addition, fluorescein, acridine orange, and the like, which are used in fluorescence spectroscopy, can be used.

In addition, the water used in this case may contain a water-soluble drug in addition to the colorant.

In addition to being able to use the colorant in the production of the medical composition, the medical composition can be colored by a method such as separately spraying the medical composition and water containing the colorant. In a case where the medical composition is, for example, the granule, since the medical composition easily absorbs water containing the colorant, a colorant that causes a coloring agent to fade in a relatively short time can be used. In addition, since the procedure can be performed without changing the colorant, for example, the medical composition and the lesion can be colored without changing the catheter, which is preferable. In this case, for example, a catheter having two flow passages can be used.

The medical composition is preferably a hemostatic agent. That is, the medical composition is preferably applied to a bleeding site, which is the surface of the inner lumen of the digestive tract of the living body. The medical composition is preferably applied as a hemostatic agent for a bleeding site on the surface of the inner lumen of the digestive tract of the living body in a treatment using an endoscope.

The medical composition as a hemostatic agent may contain raw materials other than polyacrylic acid and polyvinylpyrrolidone. Examples of the raw materials other than the polyacrylic acid and the polyvinylpyrrolidone include a drug having an effect of hemostasis, a drug having an effect of curing a lesion in a case where the hemostatic site is a lesion, and the like.

### (Hemostatic Agent)

One embodiment of the present disclosure is a hemostatic agent containing a medical composition. The hemostatic agent can stop bleeding at a bleeding site by being applied to the bleeding site on the surface of the inner lumen of the digestive tract of the living body. The medical composition is preferably sprayed on the bleeding site by a hemostatic agent spraying method using an endoscope, which has been conventionally performed.

The hemostatic agent can be applied to the surface of the inner lumen of the digestive tract of the living body by using a catheter to be inserted from a forceps port of the endoscope. The hemostatic agent is gelated due to moisture or the like of the bleeding site by spraying only the hemostatic agent, and has excellent hemostatic properties and retention properties over time. In addition, the hemostatic agent may be applied to a bleeding site by spraying the hemostatic agent and water. In this case, the water may contain a colorant or the like.

The hemostatic agent may contain raw materials other than polyacrylic acid and polyvinylpyrrolidone. Examples thereof include a drug having an effect of hemostasis, a drug having an effect of curing a lesion in a case where the hemostatic site is a lesion, and the like. Specific examples thereof include epinephrine and prednisolone.

### (Dispenser)

The dispenser according to the embodiment of the present disclosure (hereinafter, referred to as dispenser) is a dispenser that sprays the medical composition or the hemostatic agent onto the surface of the inner lumen of the digestive tract, and includes a main body and the catheter. The main body has an accommodation portion that accommodates air from the outside of the living body and that is capable of ejecting air by pressurizing the accommodated air, a container that accommodates the medical composition, and at least two check valves. The catheter has a connecting portion that is connected to the container and through which the medical composition flows. The air is ejected by the accommodation portion, whereby the medical composition can be sprayed from an end part of the catheter onto the surface of the inner lumen of the digestive tract.

In order to apply the powder to an affected part of the digestive tract via an endoscope, a spray device is required to deliver the powder body with a tube such as a catheter. However, in a case of considering the reuse of the device, gelation due to contamination after use or contact with moisture of particles remaining in the catheter is unavoidable, and there is a concern that safe and normal spraying may not be performed in a case of reuse. Therefore, it is preferable that the spray device is used as a disposable device. Further, a simple configuration in which the function of the spray device is limited as a disposable device is economically preferable for medical care.

The dispenser includes at least two check valves, and the medical composition can be suitably manually sprayed on the surface of the inner lumen of the digestive tract. For example, since the medical composition can be manually sprayed, the spray amount, spray timing, spray rate, and the like of the medical composition can be finely controlled.

In addition, in the dispenser, the accommodation portion that accommodates air can eject air with manual pressurization, and the air is automatically sucked. Therefore, a spray can containing liquefied gas or compressed gas, electricity for operating a mechanism for sucking air, and the like are not necessary. Therefore, the entire dispenser is disposable, the dispenser can be made inexpensive, there is no risk of infection or the like, and it is economically preferable for medical care.

In addition, the dispenser has at least two check valves. By combining two check valves, the medical composition can be sprayed without sucking air from the catheter. Since air is not sucked from the catheter, the backflow of the sprayed medical composition, the absorption of the moisture in the living body, and the like are suppressed, and the clogging of the catheter due to the medical composition, the gelation of the medical composition in the container due to moisture, and the like can be suppressed.

As shown in FIG. 2, a dispenser 20, which is an example of the dispenser, includes a main body 21 and a catheter 22. The main body 21 includes an accommodation portion 23, a container 24, and a check valve 25a and a check valve 25b which are two check valves.

The catheter 22 has a connector 27a at one end part. The catheter 22 is connected to the container 24, with the connector 27a, via a connector 27b provided at the end part of a connecting portion 26a of the container 24. The other end part of the catheter 22 is disposed in the living body through a forceps port of the endoscope, and the medical composition can be sprayed on the spraying portion.

A medical composition 28 is accommodated in the container 24.

In addition, the accommodation portion 23 is connected to the container 24 via the check valve 25a. The check valve 25a is provided in a connecting portion 26b that connects the accommodation portion 23 to the container 24. The check valve 25b is attached to the accommodation portion 23. A flow passage is formed in the connecting portion 26a and the connecting portion 26b, and specifically, the connecting portion 26a and the connecting portion 26b are tubes.

The main body includes at least two check valves. It is preferable that at least one of the check valves has a function of being biased in a closing direction at normal pressure and being opened in case of pressurization. It is preferable that the other check valve out of the two check valves has a function of being biased in a closing direction in case of pressurization and being opened at the normal pressure.

In a case where the main body includes at least two check valves having different functions in combination in this way, the medical composition can be sprayed by pressurization with the dispenser, and the pressurization can be stopped, so that the suction of air from the catheter can be prevented. Specifically, by combining at least two check valves having different functions, the medical composition can be sprayed by pressurization with the dispenser, and the pressurization can be stopped, so that the suction of air from the catheter can be prevented. As a result, since air is not sucked from the catheter, the backflow of the sprayed medical composition, the absorption of the moisture in the living body, and the like are suppressed, and the clogging of the catheter due to the medical composition, the gelation of the medical composition in the container due to moisture, and the like can be suppressed.

It is preferable that at least one of the check valves is a duckbill check valve. The duckbill check valve is a check valve having a function of being biased more reliably in a closing direction at normal pressure and being opened in case of pressurization. Specific examples thereof include a PP duckbill mini check valve (model number: FK212601) manufactured by ISIS Co., Ltd.

Since at least one of the at least two check valves included in the dispenser is a duckbill check valve, it is possible to more reliably suppress the suction of the air from the catheter.

The accommodation portion and the container are connected to each other by the connecting portion. It is preferable that the connecting portion, which connects the accommodation portion to the container, includes at least one check valve. It is preferable that the connecting portion includes at least one check valve that has a function of being opened in case of pressurization and being closed in a pressure state other than the pressurization, for example, negative pressure or normal pressure.

In a case of such a check valve having such a function, any check valve having any mechanism can be used. However, since it is possible to more reliably control the flow, for example, the check valve provided in the connecting portion is preferably a duckbill check valve.

As shown in FIG. 2, in the dispenser 20 which is an example of the dispenser, the connecting portion 26b includes the check valve 25a. The check valve 25a is a duckbill check valve.

It is preferable that the accommodation portion is a spherical structure including an elastic resin capable of pressurizing air by squeezing. Since the accommodation portion is a spherical structure including an elastic resin, for example, the accommodation portion can be manually squeezed, and the air can be ejected into the container by squeezing the accommodation portion.

It is preferable that the accommodation portion includes at least one check valve. In a case where the squeezing is stopped by the accommodation portion including the check valve, air is automatically sucked from the outside into the accommodation portion via the check valve.

It is preferable that the accommodation portion includes at least one check valve having a function of being closed in case of pressurization and being opened in a pressure state other than pressurization, for example, negative pressure or normal pressure. In a case of such a check valve having such a function, any check valve having any mechanism can be used. For example, it is preferable that the check valve provided in the accommodation portion is a diaphragm type check valve.

As shown in FIG. 2, in the dispenser 20 which is an example of the dispenser, the accommodation portion 23 includes the check valve 25b. The check valve 25b is a diaphragm type check valve.

As described above, since the main body of the dispenser is provided with at least two check valves having different functions at a position opposite to the catheter with respect to the container, it is possible to suppress the suction of the air from the catheter and preferably perform the spraying of the medical composition.

It is preferable that the catheter has an inner diameter of 0.5 mm to 2.5 mm and is disposed at a distal end of the main body. The inner diameter is more preferably 1.0 mm to 2.0 mm. In a case where the inner diameter of the catheter is 0.5 mm or more, the above-described medical composition can be smoothly sprayed, and in a case where the inner diameter of the catheter is 2.5 mm or less, the catheter can be inserted into a forceps port through which a treatment tool is passed in a normal endoscope.

In addition, it is preferable that the distal end of the main body on which the catheter is disposed is an end part on the opposite side to the accommodation portion of the main body. That is, it is preferable that the catheter and the accommodation portion are disposed on opposite sides to each other in the main body.

As a result, in a case where the accommodation portion is squeezed, the medical composition can be efficiently flowed into the flow passage of the catheter. The disposition of the catheter and the accommodation portion may be a disposition other than the catheter and the accommodation portion being disposed on opposite sides to each other. Due to the relationship of inserting the catheter into the endoscope forceps port, for example, the catheter and the accommodation portion may be disposed so as not to interfere with the endoscope operation, for example, at an angle of 90°.

As shown in FIG. 2, the dispenser 20 which is an example of the dispenser includes the catheter 22 at one distal end of the main body 21 via the connecting portion 26a, and includes the accommodation portion 23 at the opposite distal end.

As a result, in a case where the accommodation portion 23 is squeezed, the medical composition 28 can be efficiently sprayed from the flow passage of the catheter 22.

The catheter preferably has at least two flow passages, a flow passage through which the medical composition flows and a flow passage through which water flows. Accordingly, in a case where the medical composition is sprayed onto the surface of the inner lumen of the digestive tract of the living body, water can be sprayed onto the sprayed medical composition. The sprayed medical composition can be gelated by moisture of the mucous membrane, moisture of the blood from the bleeding site, and the like, but in some cases, the sprayed medical composition can be gelated more quickly by spraying water. In addition, as described above, the medical composition can be colored by spraying water containing a coloring agent.

The operation of the dispenser will be described. As shown in FIG. 2, in advance, the medical composition 28 is put into the container 24 of the dispenser 20 which is an example of the dispenser. Then, the catheter 22 is inserted from the forceps port of the endoscope and is disposed in the inner lumen of the digestive tract of the living body to which the medical composition is sprayed. One end of the catheter 22 is a discharge port for the medical composition.

In a case where the medical composition 28 is sprayed, the accommodation portion 23 is usually manually squeezed. Accordingly, pressure is applied to the accommodation portion 23, and the air accommodated in the accommodation portion 23 is ejected into the container 24 through the connecting portion 26b and the check valve 25a. In FIG. 2, arrows indicate a flow of air, and indicate that air flows only in one direction in each check valve.

In a case where air is ejected into the container 24, the medical composition 28 flows to the connecting portion 26a, the connector 27b, the connector 27a, and the catheter 22 through the flow passage thereof in this order, and the medical composition 28 is sprayed from one end of the catheter 22.

Next, the squeezing of the accommodation portion 23 is stopped, the pressure is released, and the check valve 25a and the check valve 25b cooperate with each other, so that only the region on the accommodation portion 23 side from the check valve 25a is in negative pressure, and the external air is sucked from the check valve 25b. As a result, the negative pressure in the accommodation portion 23 is eliminated, and air is accommodated in the accommodation portion 23.

By repeating this series of flows, the medical composition 28 can be continuously sprayed.

As in the case of the dispenser 20, the check valve 25b may be disposed at any position as long as the check valve 25b is on the accommodation portion 23 side with respect to the check valve 25a in the connecting portion 26b.

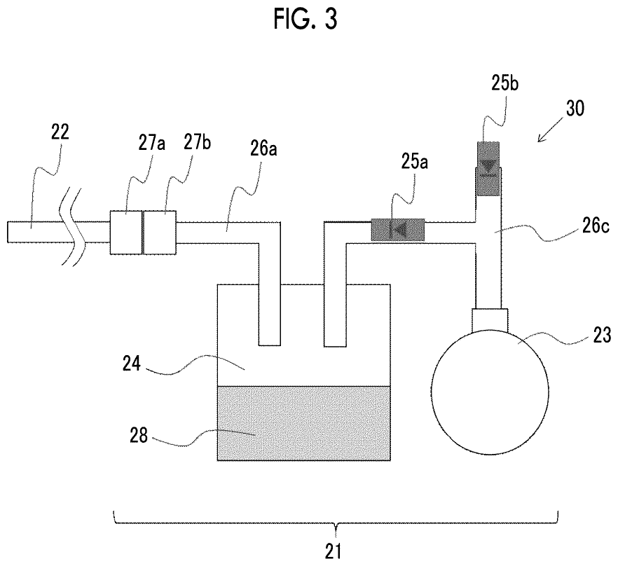

As shown in FIG. 3, in a dispenser 30 which is an example of the dispenser, the check valve 25b is disposed at one end of a connecting portion 26c branched into three parts. The container 24 can operate, for example, like a rubber ball of a dropper, and can suck air from the check valve 25b and eject air from the check valve 25a. In FIG. 3, arrows indicate a flow of air, and indicate that air flows only in one direction in each check valve.

The dispenser containing the medical composition can be distributed as a medical composition-containing dispenser, in which the medical composition is accommodated in the container of the dispenser. The medical composition-containing dispenser can be used one at a time for each procedure. The medical composition-containing dispenser is disposable, has high safety because there is no risk of infection, and is more inexpensive because electricity, a compressed spray can, or the like is not used. Thus, it is economically preferable for medical care.

### EXAMPLES

Hereinafter, the present disclosure will be described in more detail based on Examples.

The materials, the used amounts, the proportions, the treatment contents, the treatment procedures, and the like described in Examples can be appropriately changed without departing from the gist of the present disclosure. Therefore, the scope of the present disclosure should not be construed as being limited to Examples.

In the following description, “parts” and “%” are mass-based unless otherwise specified.

In addition, “Carbopol” is a registered trademark. In the following, the description that “Carbopol” is a registered trademark will be omitted.

### Examples 1 to 12

The materials shown in Table 1 were mixed and put into a fluidized bed granulator (FL-LABO manufactured by Freund Corporation) such that the final weight was 250 g. Next, pure water was sprayed onto the powder body of the powder body portion at an intake temperature of 70° C., an intake air volume of 0.3 m³/min, a spray rate of about 4 mL/min, and a spray pressure of 0.18 MPa, and then the mixture was granulated and dried under reduced pressure at 40° C. for 10 hours using a vacuum dryer to obtain a granulated product.

The average particle diameter was controlled by controlling the granulation time during which water was temporarily added.

The granulated products were all granular, and the results of measuring the average particle diameters (the volume distribution average particle diameters D50 shown above) are shown in Table 1.

The units of the compositions shown in Table 1 are % by mass with respect to the total mass of the medical composition.

In Table 1, a blank indicates that the compound is not contained.

The details of the materials shown in Table 1 are as follows.
Carbopol 971P: crosslinked powder of polyacrylic acid, manufactured by Lubrizol Corporation, particle diameter of 2.6 μm
Polyacrylic acid 1,000,000: powder of polyacrylic acid, manufactured by FUJIFILM Wako Pure Chemical Corporation, molecular weight: 1,000,000, particle diameter: 2.0 μm
PVP K90: powder of polyvinylpyrrolidone, manufactured by FUJIFILM Wako Pure Chemical Corporation, molecular weight: 360,000, particle diameter: 165 μm
PVP K30: powder of polyvinylpyrrolidone, manufactured by FUJIFILM Wako Pure Chemical Corporation, molecular weight: 40,000, particle size: 50 μm
Poly(1-vinylpyrrolidone-co-vinyl acetate): powder of copolymer of vinyl acetate and polyvinylpyrrolidone, Plasdone S-630 manufactured by Ashland Inc., molecular weight: 20,000, particle diameter: 40 μm
Blue No. 1: manufactured by Tokyo Chemical Industry Co., Ltd.
Sodium hyaluronate: manufactured by Sigma-Aldrich Co. LLC, molecular weight: 100,000, particle diameter: 10 μm

The medical composition which is the granulated product obtained in Example 2 was measured for the particle size distribution.

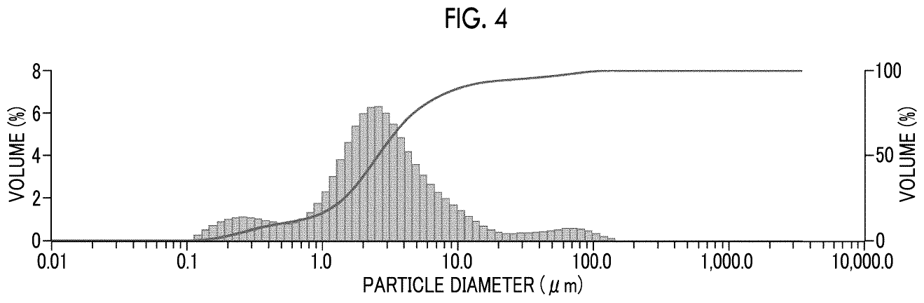

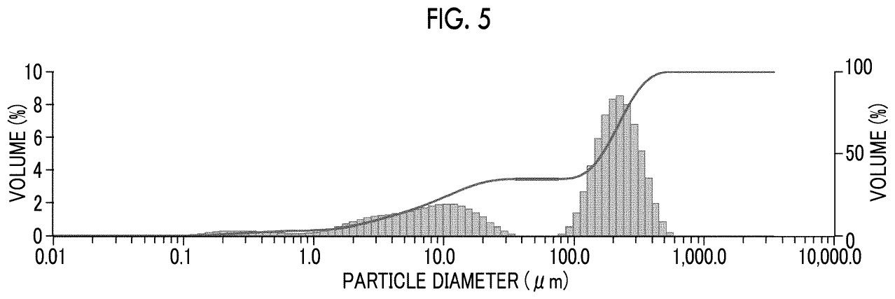

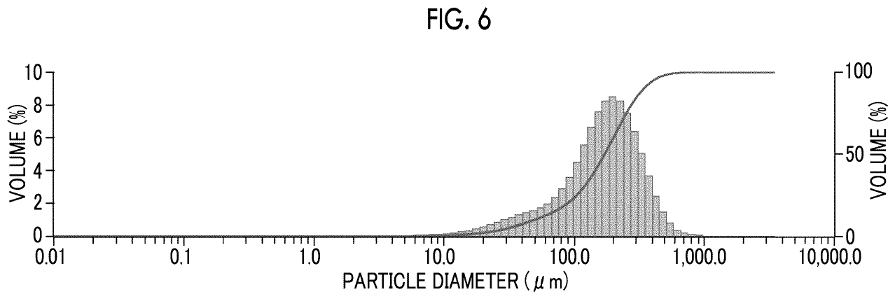

As shown in FIGS. 4 and 5, the medical composition, which is a granulated product obtained in Example 2 shown in FIG. 6, was a granulated product having a larger particle diameter than each material, had a unimodal particle size distribution, and had a relatively uniform particle diameter, as compared with the particle size distribution and average particle diameter (FIG. 4) of Carbopol 971P, which is a material of the medical composition, and the particle size distribution and average particle diameter (FIG. 5) of PVP K90, which is a material of the medical composition.

### (Evaluation)

The following tests were performed on the medical compositions obtained in Examples 1 to 12.

### <Presence or Absence of Catheter Clogging>

A catheter tube made of polytetrafluoroethylene (PTFE) having an inner diameter of 1.0 mm and an outer diameter of 1.5 mm was connected to the dispenser shown in FIG. 2, and the medical composition was sprayed.

### —Evaluation Standard—

In a case where 10 g of the medical composition obtained in each of Examples was sprayed and the powder body was sprayed to the end without clogging, it was determined that there was no clogging, and in a case where the powder body could not be sprayed in the middle, it was determined that there was clogging.

In a case where there is clogging, the user unavoidably stops the work, which is inappropriate. Therefore, the case of no clogging was regarded as pass.

### <Difficulty of Diffusion During Spraying>

In a case where the powder body is sprayed in the digestive tract, the powder is scattered in the digestive tract, and the visual field with the endoscope is blurred. Thus, there is a concern that the work of the user may be hindered. Therefore, the following evaluation was performed.

5 ml of the medical composition was enclosed in a transparent centrifuge tube bottle (Violamo centrifuge tube II 15 mL, available from AS ONE Corporation), and sensory evaluation via visual observation was performed for the floating properties of the powder in the tube in case of being vigorously stirred by hand.

In the following evaluation, 4 and 5 were regarded as pass, and 1 to 3 were regarded as fail.

### —Evaluation Standards—

5: No scattering of powder was observed immediately after stirring.
4: The powder was slightly scattered immediately after stirring, but no scattering of powder was observed within 5 seconds.
3: The powder was scattered immediately after stirring, but the powder was slightly scattered after 5 seconds.
2: The powder was scattered immediately after stirring, and the powder was scattered so much that the inside of the tube was substantially non-transparent. In a case where the time exceeded 5 seconds, a slight amount of powder was scattered.
1: The powder was scattered immediately after stirring, and the powder was scattered so much that the inside of the tube was substantially non-transparent. In a case where the time exceeded 5 seconds, a large amount of powder continued to scatter.

### <Hemostatic Property>

An abdomen of a pig was opened, and a bleeding wound part having a diameter of 6 mm and a depth of 2 mm was formed in a liver part with a biopsy trephine. 0.5 g of the medical composition was applied onto the bleeding wound part so that the bleeding wound part was covered, a coating film was formed, and the bleeding wound part was pressed with gauze wetted with saline. The state of the bleeding wound part after 1 minute, 2 minutes, 5 minutes, and 10 minutes from the application of the medical composition was visually observed, and the evaluation was carried out and summarized based on the following evaluation standards. In the following evaluation, it was determined that the hemostatic properties were good in a case where the evaluation standard was 3 to 5. 1 and 2 were regarded as fail.

### —Evaluation Standards—

5: It could be confirmed that the hemostasis was achieved in a case of observing the state of the bleeding wound part after 1 minute.
4: It could be confirmed that although the hemostasis was not achieved in a case of observing the state of the bleeding wound part after 1 minute, the hemostasis was achieved in a case of observing the state of the bleeding wound part after 2 minutes.
3: It could be confirmed that although the hemostasis was not achieved in a case of observing the state of the bleeding wound part after 2 minutes, the hemostasis was achieved in a case of observing the state of the bleeding wound part after 5 minutes.
2: It could be confirmed that although the hemostasis was not achieved in a case of observing the state of the bleeding wound part after 5 minutes, the hemostasis was achieved in a case of observing the state of the bleeding wound part after 10 minutes.
1: The hemostasis was not achieved even in a case of observing the state of the bleeding wound part after 10 minutes.

### <Retention Property Over Time>

0.5 g of the medical composition was applied to an area on a collagen casing (manufactured by Nippi, Inc.) swollen with saline, the area having a range of a diameter of 2 cm. Further, saline was gently provided on the composition to gelate the medical composition.

The medical composition attached to the collagen casing was left to stand in 100 ml of saline, a sheet was taken out over time, the attachment of the medical composition to the collagen casing was visually observed, and the score was given according to the following evaluation standards via visual observation. The higher the evaluation standard, the more preferable, and it was determined that the medical composition was practical when the score of the evaluation standard was 3 or more. 1 and 2 were regarded as fail.

### —Evaluation Standards—

5: The medical composition remained for 3 days or more.
4: The medical composition no longer remained attached to the casing in a period of 1 day or more and less than 3 days.
3: The medical composition no longer remained attached to the casing in a period of 8 hours or more and less than 1 day.
2: The medical composition no longer remained attached to the casing in a period of 1 hour or more and less than 8 hours.
1: The medical composition no longer remained attached to the casing in a short period of less than 1 hour.

| | Materials | Example 1 | Example 2 | Example 3 | Example 4 | Example 5 | Example 6 | Example 7 |
|---|---|---|---|---|---|---|---|---|
| Polyacrylic acid | Carbopol 971P | 10 | 30 | 50 | 70 | 50 | 50 | |
| | (% by mass) | | | | | | | |
| | Carbopol 934 | | | | | | | 50 |
| | (% by mass) | | | | | | | |
| | Polyacrylic acid | | | | | | | |
| | 1,000,000 | | | | | | | |
| | (% by mass) | | | | | | | |
| Polyvinylpyrrolidone | PVP K90 | 90 | 70 | 50 | 30 | | | 50 |
| | (% by mass) | | | | | | | |
| | PVP K30 | | | | | 50 | | |
| | (% by mass) | | | | | | | |
| | Poly(1-vinylpyrrolidone- | | | | | | 50 | |
| | co-vinyl acetate) | | | | | | | |
| | (% by mass) | | | | | | | |
| Other components | Blue No. 1 | | | | | | | |
| | (% by mass) | | | | | | | |
| | Sodium hyaluronate | | | | | | | |
| | (% by mass) | | | | | | | |
| Property | Average particle | 200 | 120 | 150 | 120 | 120 | 120 | 120 |
| | diameter | | | | | | | |
| | (μm) | | | | | | | |
| Evaluation | Presence or absence of | Absence | Absence | Absence | Absence | Absence | Absence | Absence |
| | catheter cloggung | | | | | | | |
| | Difficulty of diffusion | 4 | 4 | 4 | 4 | 4 | 4 | 4 |
| | during spraying | | | | | | | |
| | Hemostatic property | 3 | 5 | 5 | 5 | 5 | 4 | 5 |
| | Retention property | 3 | 5 | 5 | 5 | 4 | 3 | 5 |
| | over time | | | | | | | |

| | Materials | Example 8 | Example 9 | Example 10 | Example 11 | Example 12 |
|---|---|---|---|---|---|---|
| Polyacrylic acid | Carbopol 971P | | 50 | 50 | 50 | 25 |
| | (% by mass) | | | | | |
| | Carbopol 934 | | | | | |
| | (% by mass) | | | | | |
| | Polyacrylic acid | 50 | | | | |
| | 1,000,000 | | | | | |
| | (% by mass) | | | | | |
| Polyvinylpyrrolidone | PVP K90 | 50 | 50 | 50 | 50 | 25 |
| | (% by mass) | | | | | |
| | PVP K30 | | | | | |
| | (% by mass) | | | | | |
| | Poly(1-vinylpyrrolidone- | | | | | |
| | co-vinyl acetate) | | | | | |
| | (% by mass) | | | | | |
| Other components | Blue No. 1 | | | | 0.01 | |
| | (% by mass) | | | | | |
| | Sodium hyaluronate | | | | | 50 |
| | (% by mass) | | | | | |
| Property | Average particle | 120 | 450 | 60 | 120 | 100 |
| | diameter | | | | | |
| | (μm) | | | | | |
| Evaluation | Presence or absence of | Absence | Absence | Absence | Absence | Absence |
| | catheter cloggung | | | | | |
| | Difficulty of diffusion | 4 | 5 | 3 | 4 | 4 |
| | during spraying | | | | | |
| | Hemostatic property | 5 | 5 | 5 | 5 | 3 |
| | Retention property | 4 | 5 | 5 | 5 | 3 |
| | over time | | | | | |

### Comparative Examples 1 to 3

A granulated product was obtained using the materials shown in Table 2 in the same manner as in Examples.

The granulated product was granulated in Comparative Example 2, but was not granulated in Comparative Example 1. The results of measuring the average particle diameters (the volume distribution average particle diameters D50 shown above) are shown in Table 2.

TABLE 2

| | | Comparative | Comparative |
|---|---|---|---|
| | Materials | Example 1 | Example 2 |
| Polyacrylic acid | Carbopol 971P | 100 | |
| | (% by mass) | | |
| | Carbopol 934 | | |
| | (% by mass) | | |
| | Polyacrylic acid 1,000,000 | | |
| | (% by mass) | | |
| Polyvinylpyrrolidone | PVP K90 | | 100 |
| | (% by mass) | | |
| | PVP K30 | | |
| | (% by mass) | | |
| | Poly(1-vinylpyrrolidone-co-vinyl acetate) | | |
| | (% by mass) | | |
| Other components | Blue 1 | | |
| | (% by mass) | | |
| | Sodium hyaluronate | | |
| | (% by mass) | | |
| Property | Average particle diameter | 10 | 200 |
| | (μm) | | |
| Evaluation | Presence or absence of catheter clogging | Absence | Absence |
| | Difficulty of diffusion during spraying | 1 | 5 |
| | Hemostatic property | 3 | 1 |
| | Retention property over time | 2 | 1 |

### (Reference Examples 1 and 2)

The granulated product was obtained using the materials shown in Table 3 in the same manner as in Examples, except that the granulation method was changed.

In Reference Example 1, a powder body having a high average particle diameter was obtained by continuously providing an excessive amount of moisture as a granulation method.

In Reference Example 2, as a granulation method, PVP K90 was crushed in advance with a mortar to reduce the average particle diameter, and then granulation was performed in the same manner as in Examples, thereby obtaining a powder body having a lower average particle diameter than that of Examples.

TABLE 3

| | | Reference | Reference |
|---|---|---|---|
| | Materials | Example 1 | Example 2 |
| Polyacrylic acid | Carbopol 971P | 50 | 50 |
| | (% by mass) | | |
| | Carbopol 934 | | |
| | (% by mass) | | |
| | Polyacrylic acid 1,000,000 | | |
| | (% by mass) | | |
| Polyvinylpyrrolidone | PVP K90 | 50 | 50 |
| | (% by mass) | | |
| | PVP K30 | | |
| | (% by mass) | | |
| | Poly(1-vinylpyrrolidone-co-vinyl acetate) | | |
| | (% by mass) | | |
| Other components | Blue 1 | | |
| | (% by mass) | | |
| | Sodium hyaluronate | | |
| | (% by mass) | | |
| Property | Average particle diameter | 800 | 30 |
| | (μm) | | |
| Evaluation | Presence or absence of catheter clogging | Presence | Absence |
| | Difficulty of diffusion during spraying | 5 | 2 |
| | Hemostatic property | 5 | 5 |
| | Retention property over time | 5 | 5 |

It has been verified that the medical composition shown in Examples and Reference Examples is a medical composition excellent in both hemostatic properties and retention properties over time.

It has been further verified that the medical composition shown in Examples is less likely to cause the clogging of the catheter and the diffusion during the spraying, and in a case where the medical composition is sprayed using an endoscope, the fogging of the endoscopic visual field is suppressed.

### EXPLANATION OF REFERENCES

What is claimed is:

1. A medical composition to be applied to a surface of an inner lumen of a digestive tract of a living body,
wherein the medical composition is a powder body containing polyacrylic acid and polyvinylpyrrolidone.

2. The medical composition according to claim 1,
wherein the powder body includes a powder of the polyacrylic acid and a powder of the polyvinylpyrrolidone.

3. The medical composition according to claim 1,
wherein particles contained in a powder of the polyacrylic acid are attached to surfaces of particles contained in a powder of the polyvinylpyrrolidone to form the powder body.

4. The medical composition according to claim 1,
wherein the powder body is a granule containing a powder of the polyacrylic acid and a powder of the polyvinylpyrrolidone.

5. The medical composition according to claim 4,
wherein the granule has a volume distribution average particle diameter D50 of 50 μm to 500 μm.

6. The medical composition according to claim 1,
wherein the polyacrylic acid and the polyvinylpyrrolidone are contained at a ratio of 1:10 to 10:1 in terms of mass ratio.

7. The medical composition according to claim 1,
wherein a total content of the polyacrylic acid and the polyvinylpyrrolidone is 10% by mass to 100% by mass with respect to a total mass of the medical composition.

8. The medical composition according to claim 1,
wherein the medical composition is a hemostatic agent.

9. A hemostatic agent comprising:
the medical composition according to claim 1.

10. A dispenser that sprays the medical composition according to claim 1 onto a surface of an inner lumen of a digestive tract, the dispenser comprising:
a main body; and
a catheter,
wherein the main body has an accommodation portion that accommodates air from an outside of a living body and that is configured to eject the air by pressurizing the accommodated air, a container that accommodates the medical composition, and at least two check valves,
the catheter has a connecting portion that is connected to the container and through which the medical composition flows, and
the dispenser is configured to spray the medical composition onto the surface of the inner lumen of the digestive tract from an end part of the catheter by ejecting the air from the accommodation portion.

11. The dispenser according to claim 10,
wherein at least one of the check valves has a function of being biased in a closing direction at normal pressure and being opened in a case of pressurization.

12. The dispenser according to claim 10,
wherein at least one of the check valves is a duckbill check valve.

13. The dispenser according to claim 10,
wherein the main body includes a connecting portion that connects the accommodation portion and the container, and
the connecting portion is provided with at least one duckbill check valve.

14. The dispenser according to claim 10,
wherein the accommodation portion is a spherical structure including an elastic resin configured to pressurize the air by squeezing.

15. The dispenser according to claim 10,
wherein the catheter has an inner diameter of 0.5 mm to 2.5 mm and is disposed at a distal end of the main body.

16. The dispenser according to claim 10,
wherein the catheter has at least two flow passages, a flow passage through which the medical composition flows and a flow passage through which water flows.
